(19) United States
(12) Patent Application Publication
(10) Pub. No.: US 2018/0177963 A1
(43) Pub. Date: Jun. 28, 2018

(54) NON-INVASIVE METHOD FOR MONITORING PATIENT RESPIRATORY STATUS VIA SUCCESSIVE PARAMETER ESTIMATION

(71) Applicant: KONINKLIJKE PHILIPS N.V., EINDHOVEN (NL)

(72) Inventors: DONG WANG, SCARSDALE, NY (US); FRANCESCO VICARIO, BOSTON, MA (US); ANTONIO ALBANESE, NEW YORK, NY (US); NIKOLAOS KARAMOLEGKOS, NEW YORK, NY (US); NICOLAS WADIH CHBAT, WHITE PLAINS, NY (US)

(21) Appl. No.: 15/579,228

(22) PCT Filed: Jun. 1, 2016
(86) PCT No.: PCT/IB2016/053206

§ 371 Date: Dec. 4, 2017

Related U.S. Application Data
(60) Provisional application No. 62/169,863, filed on Jun. 2, 2015.

(51) Int. Cl. A61M 16/00; A61B 5/085; A61B 5/087; A61B 5/00

(52) U.S. Cl. CPC A61M 16/026; A61B 5/085; A61B 5/087; A61B 5/7225; A61B 5/725; A61M 2230/46; A61B 5/742; A61M 2016/0027; A61M 2016/0036; A61M 2205/502; A61M 2205/3303; A61B 5/7203

(57) ABSTRACT

A Moving Window Least Squares (MWLS) approach is applied to estimate respiratory system parameters from measured air flow and pressure. In each window, elastance Eᵣₛ (or resistance Rᵣₛ) is first estimated, and a Kalman filter may be applied to the estimate. This is input to a second estimator that estimates R (or E), to which a second Kalman filter may be applied. Finally, the estimated Eᵣₛ and Rᵣₛ are used to calculate muscle pressure Pₘᵤₛ(t) in the time window. A system comprises a ventilator (100), an airway pressure sensor (112), and an air flow sensor (114), and a respiratory system analyzer (120) that performs the MWLS estimation. Estimated results may be displayed on a display (110) of the ventilator or of a patient monitor. The estimated Pₘᵤₛ(t) may be used to reduce patient-ventilator dyssynchrony, or integrated to generate a Work of Breathing (WOB) signal for controlling ventilation.

### FIELD

The following relates generally to systems and methods for monitoring and characterizing respiratory parameters during patient ventilation. It finds particular application in a system to provide real-time diagnostic information to a clinician to personalize a patient's ventilation strategy and improve patient outcomes and will be described with particular reference thereto. However, it is to be understood that it also finds application in other usage scenarios and is not necessarily limited to the aforementioned application.

### BACKGROUND

Real-time assessment of the respiratory system's parameters (resistance Rᵣₛ and compliance Cᵣₛ) and patient's inspiratory effort (respiratory muscle pressure Pₘᵤₛ(t)) provides invaluable diagnostic information for clinicians to optimize ventilation therapy.

A good Pₘᵤₛ(t) estimation can be used to quantify patient's inspiratory effort and select the appropriate level of ventilation support in order to avoid respiratory muscle atrophy and fatigue. Moreover, the estimated Pₘᵤₛ(t) waveform can also be used for triggering and cycling off the ventilator so as to reduce patient-ventilator dyssynchrony. Estimates of Rᵣₛ and Cᵣₛ are also important, as they provide quantitative information to clinicians about the mechanical properties of the patient's respiratory system and they can be used to diagnose respiratory diseases and better select the appropriate ventilator settings.

Pₘᵤₛ(t) is traditionally estimated via esophageal pressure measurement. This technique is invasive, in the sense that a balloon needs to be inserted inside the patient's esophagus, and moreover, not reliable when applied during long periods in intensive care conditions.

Another option to estimate Pₘᵤₛ(t) is to calculate it based on the Equation of Motion of the Lungs. Assuming Rᵣₛ and Cᵣₛ are known, it is indeed possible to estimate Pₘᵤₛ(t) via the following equation, known as the Equation of Motion of the lungs:

$${P}_{y} ( t ) = {R}_{rs} \dot{V} ( t ) + ( \frac{1}{{C}_{rs}} ) V ( t ) + {P}_{mus} ( t ) + {P}_{0} \qquad ( 1 )$$

where P_{y}(t) is the pressure measured at the Y-piece of the ventilator, \dot{V}(t) is the flow of air into and out of the patient's respiratory system (measured again at the Y-piece), V(t) is the net volume of air delivered by the ventilator to the patient (measured by integrating the flow signal \dot{V}(t) over time), P₀ is a constant term to account for the pressure at the end of expiration (needed to balance the equation but not interesting per se) and will be considered as part of Pₘᵤₛ(t) in the following discussion. However, Rᵣₛ and Cᵣₛ have to be measured or estimated first.

Rᵣₛ and Cᵣₛ may be estimated by applying the flow-interrupter technique (also called End Inspiratory Pause, EIP), which however interferes with the normal operation of the ventilator, or under specific conditions where the term Pₘᵤₛ(t) can be “reasonably” assumed to be zero (i.e. totally unload patient's respiratory muscles). These conditions include: periodic paralysis in which the patient is under Continuous Mandatory Ventilation (CMV); periodic high pressure support (PSV) level; specific portions of each PSV breath that extend both during the inhalation and the exhalation phases; and exhalation portions of PSV breaths where the flow signal satisfies specific conditions that are indicative of the absence of patient's inspiratory effort.

Rᵣₛ and Cᵣₛ estimation using the EIP maneuver has certain drawbacks and relies on certain assumptions. The EIP maneuver interrupts the normal ventilation needed by the patient. It also assumes that patient respiratory muscles are fully relaxed during the EIP maneuver in order for the Rᵣₛ and Cᵣₛ computation to be valid. Further, the Rᵣₛ and Cᵣₛ estimates obtained via the EIP maneuver, which affect the estimate of Pₘᵤₛ(t) on the following breath, are assumed to be constant until the next EIP maneuver is executed, so that continuous and real-time estimates of Rᵣₛ and Cᵣₛ are not obtained. In practice, changes in patient's conditions can occur in between two consecutive EIP maneuvers, and this would jeopardize the estimate of Pₘᵤₛ(t). A further disadvantage is that the static maneuver (EIP) is performed in a specific ventilation mode (Volume Assisted Control, VAC) and the obtained values for R and C might not be representative of the true values that govern the dynamics of the lungs in other ventilation modes, such as Pressure Support Ventilation (PSV). Therefore, the accuracy of Pₘᵤₛ(t) computed via equation (1) during PSV operation can be compromised.

The above mentioned estimation methods operate on the assumption that Pₘᵤₛ(t) is negligible. Implementation of this assumption can be problematic in clinical settings. For example, imposing periodic paralysis and CMV on a patient is generally not clinically feasible. Similarly, imposing periodic high PSV interferes with the normal operation of the ventilator and may not be beneficial to the patient. The assumption of negligible Pₘᵤₛ(t) during PSV breaths is debatable, especially during the inhalation phase. Approaches which operate on a chosen portion of the respiration cycle also limit the fraction of data points that are used in the fitting procedure, which makes the estimation results more sensitive to noise.

In the following, non-invasive methods are disclosed for monitoring patient respiratory status via successive parameter estimation, which overcome various foregoing deficiencies and others.

### SUMMARY

In accordance with one aspect, a medical ventilator device is described. The device includes a ventilator configured to deliver ventilation to a ventilated patient, a pressure sensor configured to measure the airway pressure P_{y}(t) at a Y-piece of the ventilator and an air flow sensor configured to measure the air flow \dot{V}(t) into and out of the ventilated patient at the Y-piece of the ventilator. The device also comprises a respiratory system monitor comprising a microprocessor configured to estimate respiratory parameters of the ventilated patient using moving window least squares (MWLS) estimation including (i) respiratory system elastance or compliance (Eᵣₛ or Cᵣₛ), (ii) respiratory system resistance (Rᵣₛ), and (iii) respiratory muscle pressure (Pₘᵤₛ(t)).

In accordance with another aspect, a method comprises: ventilating a patient using a ventilator; during the ventilating, measuring airway pressure P_{y}(t) and air flow \dot{V}(t) of air into and out of the patient; using a microprocessor, applying moving window least squares (MWLS) estimation to estimate (i) the patient's respiratory system elastance or compliance Eᵣₛ or Cᵣₛ, (ii) the patient's respiratory system resistance Rᵣₛ, and (iii) the patient's respiratory muscle pressure Pₘᵤₛ(t); and displaying on a display one or more of the respiratory parameters of the patient estimated by applying MWLS estimation.

One advantage resides in providing a non-invasive method for monitoring patient respiratory status via successive parameter estimation including resistance, compliance, and respiratory muscle pressure.

Another advantage resides in providing a ventilator with improved data analysis.

Still further advantages of the present invention will be appreciated to those of ordinary skill in the art upon reading and understand the following detailed description. It is to be appreciated that none, one, two, or more of these advantages may be achieved by a particular embodiment.

### BRIEF DESCRIPTION OF THE DRAWINGS

The disclosure may take form in various components and arrangements of components, and in various steps and arrangement of steps. The drawings are only for purposes of illustrating the preferred embodiments and are not to be construed as limiting the invention.

FIG. 1 illustrates a ventilation system for use on a patient with the proposed ventilation estimation scheme.

FIG. 2 illustrates a block diagram of the described estimation scheme.

FIG. 3 illustrates a moving window least squares algorithm for the Eᵣₛ estimation.

FIG. 4 illustrates a moving window least squares algorithm example of the polynomial order of the local Pₘᵤₛ(t) waveform.

FIG. 5 illustrates the maximum ratio combination for the MWLS Rᵣₛ estimation results.

### DETAILED DESCRIPTION

The following relates to characterization of respiratory parameters during patient ventilation and in particular to the respiratory muscle pressure Pₘᵤₛ(t), respiratory resistance Rᵣₛ, and respiratory compliance Cᵣₛ or elastance Eᵣₛ=1/Cᵣₛ. In principle, these parameters can be estimated using the Equation of Motion of the Lungs (Equation (1)), which relates these parameters to the pressure P_{y}(t) at the ventilator mouthpiece and the air flow \dot{V}(t), along with the air volume in the lungs V(t)=∫\dot{V}(t)dt. In practice, because the respiratory muscle pressure Pₘᵤₛ(t) varies over time, estimating Pₘᵤₛ(t), Rᵣₛ, and Eᵣₛ jointly using the Equation of Motion of the Lungs is generally underdetermined and cannot be analytically solved. Various approaches to dealing with this include measuring additional information using invasive probes, or creating “special case” circumstances by operations such as interrupting normal breathing. Invasive probes have apparent disadvantages, while techniques that rely upon manipulating normal patient breathing cannot provide continuous monitoring of normal respiration and may be detrimental to the patient.

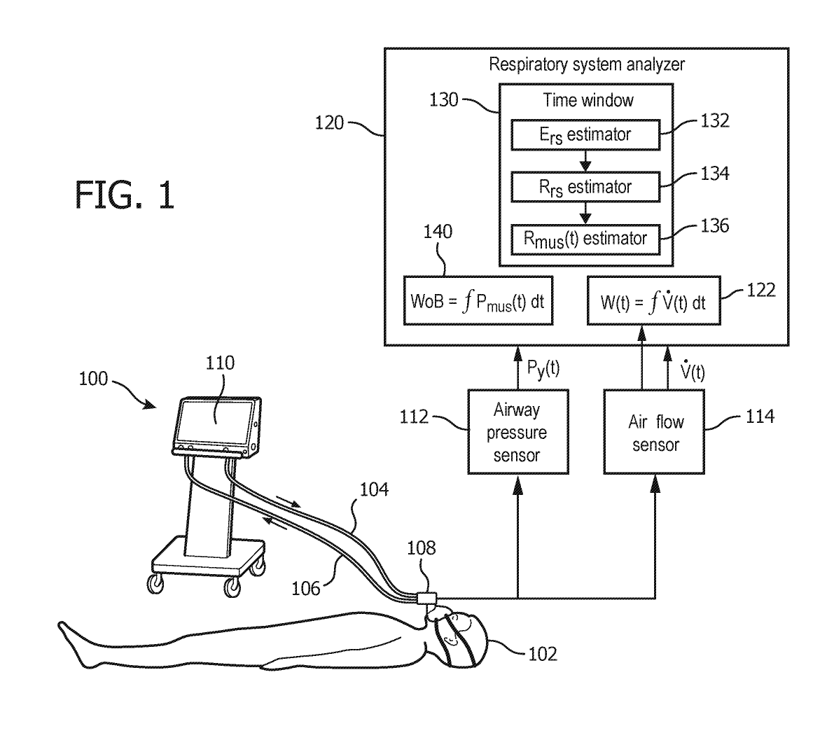

With reference to FIG. 1, a medical ventilator system includes a medical ventilator 100 that delivers air flow at a positive pressure to a patient 102 via an inlet air hose 104. Exhaled air returns to the ventilator 100 via an exhalation air hose 106. A Y-piece 108 of the ventilator system serves to couple air from the discharge end of the inlet air hose 104 to the patient during inhalation and serves to couple exhaled air from the patient into the exhalation air hose 106 during exhalation. Note the Y-piece 108 is sometimes referred to by other nomenclatures, such as a T-piece. Not shown in FIG. 1 are numerous other ancillary components that may be provided depending upon the respiratory therapy being received by the patient 102. Such ancillary components may include, by way of illustration: an oxygen bottle or other medical-grade oxygen source for delivering a controlled level of oxygen to the air flow (usually controlled by the Fraction of Inspired Oxygen (FiO₂) ventilator parameter set by the physician or other medical personnel); a humidifier plumbed into the inlet line 104; a nasogastric tube to provide the patient 102 with nourishment; and so forth. The ventilator 100 includes a user interface including, in the illustrative example, a touch-sensitive display component 110 via which the physician, respiratory specialist, or other medical personnel can configure ventilator operation and monitor measured physiological parameters and operating parameters of the ventilator 100. Additionally or alternatively, the user interface may include physical user input controls (buttons, dials, switches, et cetera), a keyboard, a mouse, audible alarm device(s), indicator light(s), or so forth. It is also noted that the illustrative ventilator 100 is merely an illustrative example.

The illustrative ventilator 100 is a dual-limb ventilator with proximate sensors. However, the disclosed patient respiratory status monitoring techniques may be employed in conjunction with substantially any type of ventilator, such as with a single-limb or dual-limb ventilator, a ventilator having valves or blower, a ventilator with an invasive coupling to the patient (e.g. via a tracheostomy or endotracheal tube) or a ventilator with a noninvasive coupling to the patient (e.g. using a facial mask), a ventilator with proximal sensors for measuring pressure and flow as illustrated or a ventilator without such proximal sensors that relies upon sensors in the ventilator unit, or so forth.

With continuing reference to FIG. 1, the patient 102 is monitored by various physiological parameter sensors. In particular, FIG. 1 illustrates two such sensors: an airway pressure sensor 112 that measures pressure P_{y} (t) at the coupling to the patient (usually measured at the Y-piece 108, hence P_{y} (t)) and an air flow sensor 114 that measures air flow \dot{V}(t) to or from the patient (also usually measured at the Y-piece 108). The sensors 112, 114 may be integrated into the Y-piece 108, interposed on the air lines 104, 106, or integrated into the ventilator 100. During mechanical ventilation, other physiological parameters may be monitored by suitable sensors, such as heart rate, respiratory rate, blood pressure, blood oxygenation (e.g. SpO₂), respiratory gases composition (e.g. a capnograph measuring CO₂ in respiratory gases), and so forth. Other physiological parameters may be derived from directly measured physiological parameters.

The system further includes a respiratory system analyzer 120 comprising a microprocessor, microcontroller, or other electronic data processing device programmed to process input data including the airway pressure P_{y} (t) and air flow \dot{V}(t) to generate information about the patient respiratory system parameters: resistance Rᵣₛ, compliance Cᵣₛ (or, equivalently, elastance Eᵣₛ=1/Cᵣₛ), and the patient's inspiratory effort characterized as a function of time by the respiratory muscle pressure Pₘᵤₛ(t). These parameters are determined as a function of time, in real-time, by evaluating the Equation of Motion of the Lungs (Equation (1)) using moving window least squares estimation (MWLS) applied to the airway pressure P_{y} (t) and air flow \dot{V}(t) along with the air volume V(t)=∫\dot{V}(t)dt determined from \dot{V}(t) by an air flow integrator 122. (Alternatively, a dedicated air volume sensor may be employed). To overcome the underdetermined nature of Equation (1), the MWLS estimation is performed using successive estimation of: (1) the elastance or compliance (Eᵣₛ or Cᵣₛ) parameter via an Eᵣₛ estimator 132; followed by (2) estimation of the resistance (Rᵣₛ) parameter via an Rᵣₛ estimator 134; followed by (3) estimation of the respiratory muscle pressure (Pₘᵤₛ(t)) parameter via a Pₘᵤₛ(t) estimator 136.

These successive estimators 132, 134, 136 are applied within the time window 130 which is generally of duration two seconds or less, and more preferably of duration one second or less, and in an illustrative example of duration 0.6 seconds with data sampling at 100 Hz so that the time window contains 60 samples. An upper limit on the duration of the time window is imposed by the respiration rate, which for a normal adult is typically 12 to 20 breaths per minute corresponding to a breathing cycle of duration 3-5 seconds. The duration of the time window 130 is preferably a fraction of the breathing cycle duration so that the parameters Eᵣₛ and Rᵣₛ can be reasonably assumed to be constant within each time window 130, and variation of Pₘᵤₛ(t) within each time window 130 can be represented using a relatively simple approximation function (e.g. a low-order polynomial in the illustrative examples disclosed herein).

The estimators 132, 134, 136 are successively applied within each time window 130, and for each successive (and partially overlapping) time interval 130 (hence the term “moving” time window), to provide estimation of Eᵣₛ, Rᵣₛ, and Pₘᵤₛ(t) in real time. In the illustrative examples, the values of Eᵣₛ and Rᵣₛ are assumed to be constant within each time window 130, so that the estimation of these parameters is in real-time with a time resolution comparable to the duration of the time window 130, e.g. two second or less in some embodiments, or more preferably one second or less, and 0.6 seconds in the illustrative examples. If successive time windows partially overlap, this can further improve the effective time resolution. The real-time estimation of Pₘᵤₛ(t) can be of higher temporal resolution than Eᵣₛ and Rᵣₛ, since variation of Pₘᵤₛ(t) with time within the time window 130 is, in the illustrative examples, modeled by a low-order polynomial function of time.

The approach disclosed herein leverages the recognition that, of the three parameters being estimated, the elastance/compliance (Eᵣₛ or Cᵣₛ) generally varies most slowly over time. In the Equation of Motion of the Lungs (Equation (1)), Eᵣₛ is the coefficient of the air volume V(t) which, as an integral, varies slowly over time. The next most slowly varying parameter is generally the resistance Rᵣₛ, which is the coefficient of the air flow \dot{V}(t). Finally, the respiratory muscle pressure Pₘᵤₛ(t) has the potential to vary most rapidly over time as it changes in response to the patient actively inhaling and exhaling. In view of this, the illustrative examples of the Pₘᵤₛ(t) estimator 136 do not assume Pₘᵤₛ(t) is a constant within the time window 130, but instead employ a low-order approximation polynomial function. Instead of a low-order polynomial approximation of Pₘᵤₛ(t) within the time window 130, in other contemplated embodiments some other parameterized function of time is contemplated, such as a spline function.

With continuing reference to FIG. 1, the outputs Eᵣₛ (or Cᵣₛ), Rᵣₛ, and Pₘᵤₛ(t) can be used for various purposes. In one application, one or more of the estimated parameters may be displayed on the display component 110 of the ventilator 100, for example as a numeric real-time value and/or as a trend line plotted as a function of time. Typically, the respiratory elastance or compliance (Eᵣₛ or Cᵣₛ) and the respiratory resistance (Rᵣₛ) are of most interest to the clinician and are suitably displayed and/or trended. The respiratory muscle pressure Pₘᵤₛ(t) is a waveform acquired as a function of time in real-time during normal clinically operative mechanical ventilation accordingly, Pₘᵤₛ(t) can be used by the ventilator 100 for triggering and cycling off the mechanical ventilation so as to reduce patient-ventilator dyssynchrony (that is, to synchronize application of positive pressure by the ventilator 100 with the inhalation portion of the patient's respiratory muscle action).

In some embodiments, a work of breathing (WoB) estimator 140 integrates the respiratory muscle pressure Pₘᵤₛ(t) over volume, i.e. WoB=∫Pₘᵤₛ (t)dV(t). The WoB is a metric of how much effort the patient 102 is applying to breathe on his or her own. The WoB may be displayed and/or trended on the display component 110 to provide the clinician with useful information for setting ventilator pressure settings in ventilation modes such as Pressure Support Ventilation (PSV). Moreover, since the WoB estimator 140 provides WoB in real-time (e.g. with a time lag and resolution on the order of a second or less in some embodiments) the ventilator 100 optionally employs the WoB as a feedback control parameter, e.g. adjusting controlled ventilator settings to maintain the WoB at a constant set-point value. For example, if the WoB increases, this implies the patient 102 is struggling to breathe and accordingly the positive pressure applied by the ventilator 100 in PSV mode should be increased to provide the struggling patient with increased respiration assistance.

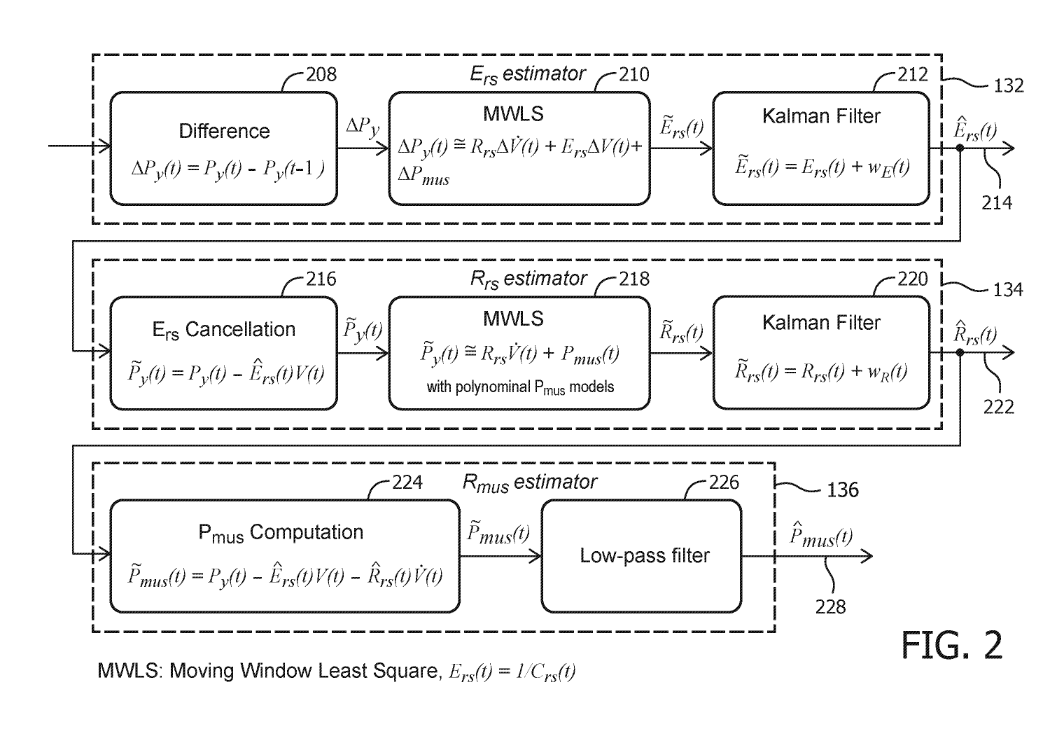

With reference to FIG. 2, some illustrative embodiments of the successive estimators 132, 134, 136 are described. successive estimation of the parameters Eᵣₛ, Rᵣₛ, and Pₘᵤₛ(t) over a time window of a fraction of a second over which the parameters Eᵣₛ and Rᵣₛ are assumed to be constant is shown. In the first pass (performed by the Eᵣₛ estimator 132), all three parameters Eᵣₛ, Rᵣₛ, and ΔPₘᵤₛ(t) are assumed to be constant over the time window 130 and are computed simultaneously—but only the estimated Êᵣₛ is retained from this first pass. (In notation used herein, the overscript “hat”, i.e. \hat{p}, is used to indicate the estimated value of parameter p.) In a second pass (performed by the Rᵣₛ estimator 134), the contribution of the now known (estimated) Êᵣₛ is removed by subtraction, and the remaining portion of the Equation of Lung Motion is fitted for Rᵣₛ and Pₘᵤₛ(t), the latter being approximated using a low order polynomial (n=0, 1, or 2). In experiments, it was found that the best choice of polynomial order is dependent upon the respiratory phase at which the time window 130 is located due to possible overfitting—as respiratory phase is not known a priori, in illustrative embodiments disclosed herein a weighted combination of polynomials of zeroeth, first, and second order is used. The output of the Rᵣₛ estimator 134 is the estimated value of the respiratory resistance, i.e. \hat{R}ᵣₛ. Finally, in a third pass (performed by the Pₘᵤₛ(t) estimator 136), the contribution of the now known (estimated) \hat{R}ᵣₛ is removed by further subtraction, and the remaining portion of the Equation of Lung Motion is directly fitted to obtain the estimated respiratory muscle pressure, i.e. \hat{P}ₘᵤₛ (t).

With continuing reference to FIG. 2, the illustrative Eᵣₛ estimator 132 is further described. At 208 a difference operation is performed on the airway pressure P_{y}(t) and the output ΔP_{y}(t) is calculated as ΔP_{y}(t)=P_{y} (t)−P_{y} (t−1). A Moving Window Least Squares (MWLS) estimator is used to at 210 to continuously estimate Eᵣₛ(t)—which is the respiratory system's elastance, Eᵣₛ(t)=1/Cᵣₛ(t)—and is based on the following difference equation:

ΔP_{y}(t)≅RᵣₛΔ\dot{V}(t)+EᵣₛΔV(t)+ΔPₘᵤₛ

It should be noted that Eᵣₛ(t) is estimated as a function of time insofar as the estimate Êᵣₛ is generated for each time window 130, so that the time function Êᵣₛ(t) is generated as the value Êᵣₛ for successive time windows 130 as successive (partially overlapping) time windows are applied over time. However inside each time window 130, ΔPₘᵤₛ(t), the difference signal of the Pₘᵤₛ(t) waveform, and the parameters Rᵣₛ(t) and Eᵣₛ(t) are modeled as constants and jointly estimated by a least squares minimization method. For the Eᵣₛ estimator 132, only the estimate of Eᵣₛ(t), namely Êᵣₛ, is used (after filtering by a Kalman filter 212 in the illustrative example of FIG. 2), while the other estimation outputs are discarded. Moreover, the Eᵣₛ estimator 132 also calculates the variance of the estimate Êᵣₛ, denoted herein as δ_{Ê}_{ᵣₛ}.

The input to the MWLS estimator 210 is the difference signal of P_{y}(t), that is, ΔP_{y}(t), which is output by the difference operation 208. Based on the equation of the motion (Equation (1)), ΔP_{y}(t) can be modeled as:

ΔP_{y}(t)≅Rᵣₛ(t)Δ\dot{V}(t)+Eᵣₛ(t)ΔV(t)+ΔPₘᵤₛ(t)

where Δ\dot{V}(t)=\dot{V}(t)−\dot{V}(t−1) is the flow difference signal, ΔV(t)=V(t)−V(t−1)=\dot{V}(t)T is the volume difference signal (where T is the sampling time interval, e.g. sampling at 100 Hz corresponds to T=0.01 sec), and ΔPₘᵤₛ(t)=Pₘᵤₛ(t)−Pₘᵤₛ(t−1) is the Pₘᵤₛ(t) difference signal.

In the following, the size (or duration) of the sliding time window 130 is denoted as L, which is optionally a system parameter that can be set by the user. The sliding window at a current time t spans the interval [t−L+1, t]. For the MWLS estimator 210, the Pₘᵤₛ(t) difference signal, ΔPₘᵤₛ(t), in the sliding window is modeled as a constant, ΔPₘᵤₛ. It is further assumed that Rᵣₛ and Eᵣₛ are constant in the sliding time window 130. Therefore, the equation for ΔP_{y}(t) becomes:

ΔP_{y}(t)≅RᵣₛΔ\dot{V}(t)+EᵣₛΔV(t)+ΔPₘᵤₛ

At time t, the MWLS algorithm 210 uses the input signals in the sliding window 130, that is to say, the samples ΔP_{y}(n) and \dot{V}(n) in the interval t−L+1≤n≤t, to estimate Rᵣₛ, Eᵣₛ, and ΔPₘᵤₛ jointly, but only the Eᵣₛ estimate Êᵣₛ is used in the subsequent operations (i.e. the subsequent estimators 134, 136).

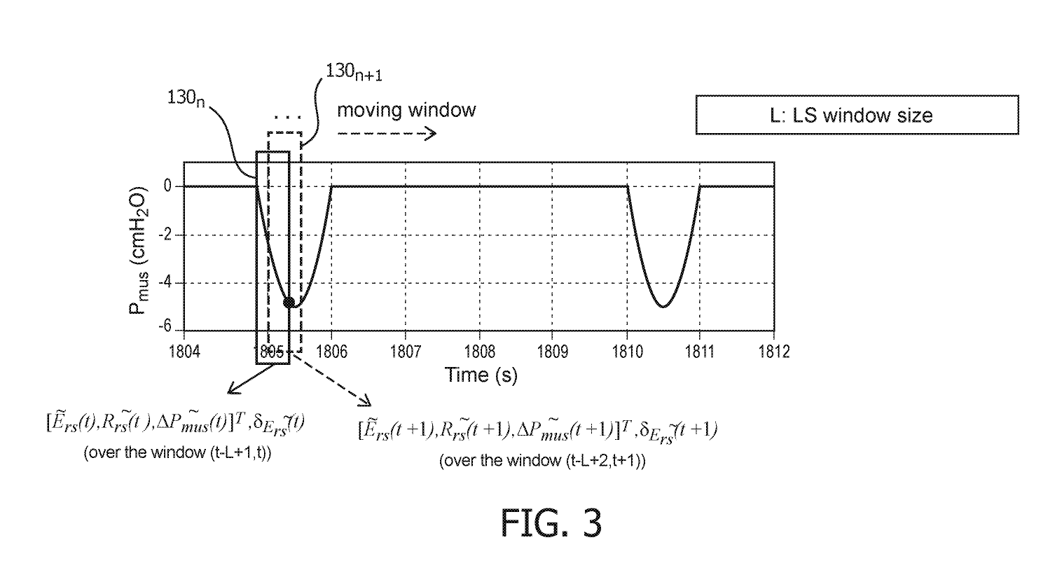

As further shown in FIG. 3, specifically, at time t, the MWLS formulation solves the least square problem 300 based on the equation described above:

At time t,

ΔP_{y}(t)≅RᵣₛΔ\dot{V}(t)+EᵣₛΔV(t)+ΔPₘᵤₛ

[\tilde{E}ᵣₛ,\tilde{R}ᵣₛ,Δ\tilde{P}ₘᵤₛ]^{T}=(X^{T}X)⁻¹X^{T}Y

Y=[ΔP_{y}(t),ΔP_{y}(t−1), . . . ,ΔP_{y}(t−L+1)]^{T}

X=[x(t),x(t−1), . . . ,x(t−L+1)]^{T}

x(t)=[ΔV(t),Δ\dot{V}(t),1]^{T}

δ_{\tilde{E}}_{ᵣₛ}=(X^{T}X)⁻¹(1,1)*δ_{ΔP}_{_{y}}

Moreover, the variance of the Eᵣₛ estimate, δ_{\tilde{E}}_{ᵣₛ}, is also calculated, where δ_{ΔP}_{_{y}} is the least square residual variance,

δ_{ΔP}_{_{y}}=(Y−X[\tilde{E}ᵣₛ,\tilde{R}ᵣₛ,Δ\tilde{P}ₘᵤₛ]^{T})^{T}(Y−X[\tilde{E}ᵣₛ,\tilde{R}ᵣₛ,Δ\tilde{P}ₘᵤₛ]^{T})/L

As indicated in FIG. 3, the MWLS estimation 210 is performed continuously as the moving window moves forward, e.g. window 310ₙ succeeded by next window 310ₙ₊₁, and so forth. Since the MWLS method is sensitive to the P_{y} measurement noise and the modelling error, only the estimate of Eᵣₛ, \tilde{E}ᵣₛ, is retained by the Eᵣₛ estimator 132 and the other estimate outputs (e.g. \tilde{R}ᵣₛ and Δ\tilde{P}ₘᵤₛ) are discarded.

To further improve the Eᵣₛ estimation performance, a Kalman filter 212 is optionally used to reduce the Eᵣₛ estimation error. As previously mentioned, the respiratory system elastance, Eᵣₛ, typically does not change rapidly as a function of time. The Kalman filter 212 is used to filter the estimation noise in \tilde{E}ᵣₛ(t) and improve the Eᵣₛ(t) estimation results. The inputs to the Kalman filter 212 are \tilde{E}ᵣₛ (t) and δ_{Ê}_{ᵣₛ}(t). The output of the Kalman filter 214 is the final estimate of Eᵣₛ(t), notated herein as Êᵣₛ(t), and \tilde{E}ᵣₛ(t)=Eᵣₛ(t)+ω_{E} (t) where ω_{E} (t) is a noise or uncertainty metric. The above model assumes that \tilde{E}ᵣₛ (t) is an unbiased estimate of Eᵣₛ(t) that has a noise term ω_{E}(t)˜N(0, δ_{\tilde{E}}_{ᵣₛ}(t)).

The Kalman filter can be designed to reduce the MWLS estimation noise based on the following assumptions: (1) a state process equation where Eᵣₛ changes slowly and can be modelled as a random walk, i.e. Eᵣₛ(t)=Eᵣₛ(t−1)+ω_{E}(t), ω_{E}(t)˜N (0, δ_{E}); and (2) an observation equation where the MWLS estimate \tilde{E}ᵣₛ(t) can be modelled as \tilde{E}ᵣₛ(t)=Eᵣₛ(t)+ω_{LE} (t), ω_{LE}(t)˜N(0, δ_{\tilde{E}}_{ᵣₛ}(t)). A standard Kalman filter can be implemented with A=1, B=0, Q=δ_{E}, H=1, and R=δ_{\tilde{E}rs}(t). The Kalman filter has certain advantages, including computationally efficient implementation in the context of a sliding time window, intuitive operation and output of weighted averages. The parameter δ_{E} is an algorithm parameter that controls the average window length.

With continuing reference to FIGS. 1 and 2, the final output Êᵣₛ (t) 214 of the Eᵣₛ estimator 132 is utilized by the succeeding Rᵣₛ estimator 134 in performing the Rᵣₛ(t) estimation. To estimate Rᵣₛ(t), the elastic pressure component EᵣₛV(t) is cancelled out of P_{y} (t) using Êᵣₛ(t). This Eᵣₛ cancellation operation 216 can be expressed as:

\tilde{P}_{y}(t)=P_{y}(t)−Êᵣₛ(t)V(t)

The Eᵣₛ cancellation 216 removes one unknown (Eᵣₛ) from the Equation of Motion of the Lungs, and thus simplifies the Rᵣₛ estimation. Assuming the estimation Êᵣₛ(t) output by the Eᵣₛ estimator 132 is correct and the elastic pressure component is perfectly cancelled, the MWLS operation 218 of the Rᵣₛ estimator 134 optimizes the equation:

\tilde{P}_{y}(t)=≅Rᵣₛ\dot{V}(t)+Pₘᵤₛ(t)

Using the Moving Window Least Squares (MWLS) estimator 218, the respiratory resistance Rᵣₛ is estimated.

In the Eᵣₛ estimator MWLS operation 210 of the Eᵣₛ estimator 132, the respiratory muscle pressure Pₘᵤₛ(t) is indirectly estimated as a linear function of t since the difference of Pₘᵤₛ(t), namely ΔPₘᵤₛ(t), is estimated as a constant value ΔPₘᵤₛ for each time window. However, it has been found herein that this estimate is unduly coarse in the case of the MWLS operation 218 of the Rᵣₛ estimator 134, and that significantly improved estimation of the respiratory resistance Rᵣₛ is provided if the time dependence of the respiratory muscle pressure Pₘᵤₛ(t) is adaptively modeled in the MWLS operation 218. In illustrative examples herein, Pₘᵤₛ(t) is modeled using a low order polynomial, e.g. of order 0 (constant value), 1 (linear), or 2 (quadratic). The order of the Pₘᵤₛ(t) polynomial function, M, can significantly change the estimation performance.

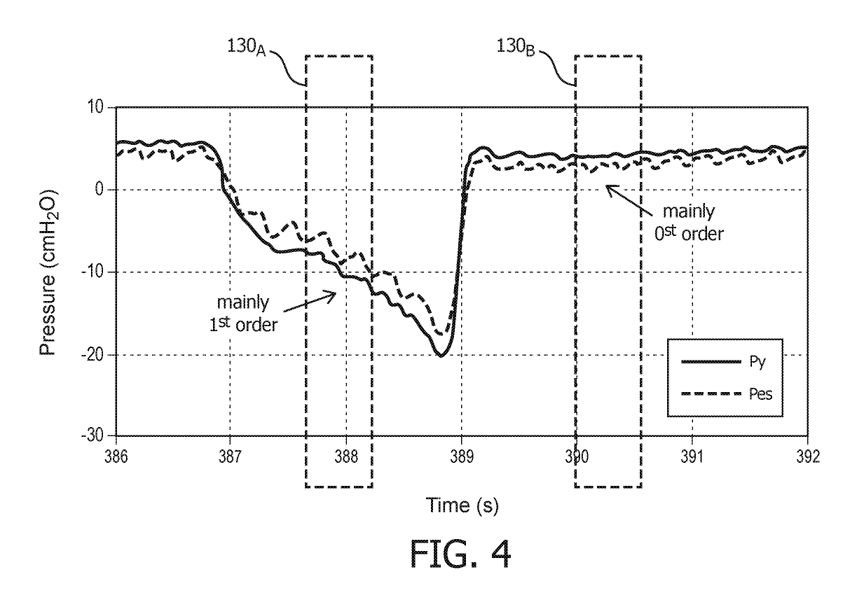

With brief reference to FIG. 4, moreover, the optimal order M of the polynomial used to model Pₘᵤₛ(t) depends on the position of the moving window 130 within the respiratory cycle. In illustrative FIG. 4, the first time window 130_{A} is located at a respiratory phase for which a first order (M=1) polynomial is an effective model of Pₘᵤₛ(t); whereas, for the respiratory phase at which the second time window 130_{B} is located a zeroeth order (M=0) polynomial is effective. However, the respiratory phase at which the current time window 130 resides is generally not an input to the Rᵣₛ estimator 134.

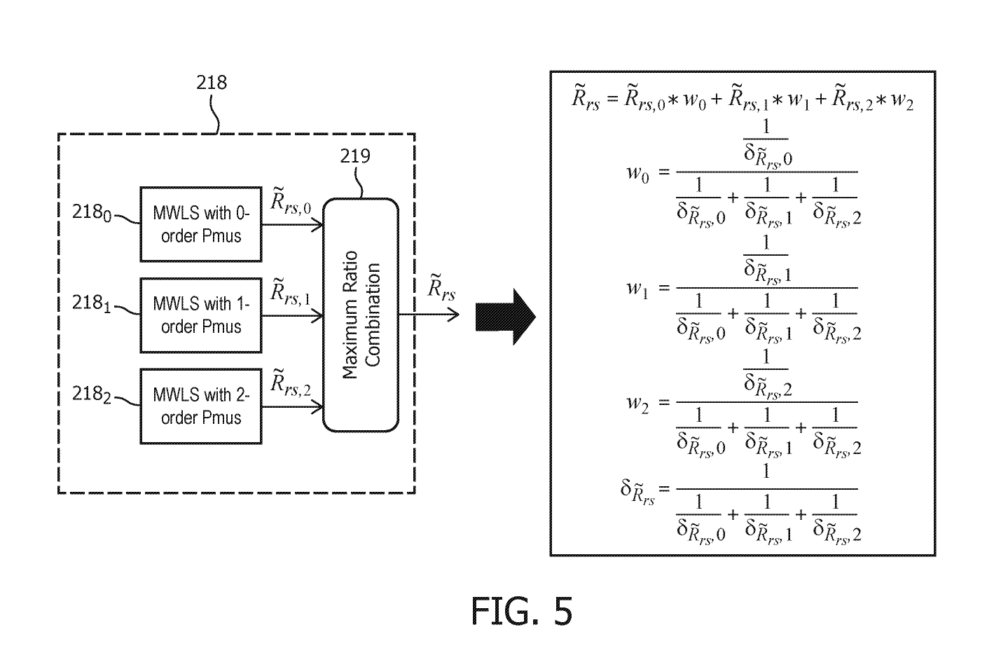

With brief reference to FIG. 5, for the Rᵣₛ MWLS 218, the Pₘᵤₛ(t) waveform is modeled as an M^{th}-order polynomial function (M>=0), i.e. Pₘᵤₛ(t)=a₀+a₁t+ . . . +a_{M}t^{M}, and the Rᵣₛ(t) parameter is assumed to be constant. (While a polynomial model of Pₘᵤₛ(t) is described herein for illustration, other models comprising a parameterized function of time such as a spline model are also contemplated.) To accommodate the differences in optimal polynomial order over the respiratory cycle, the Rᵣₛ MWLS estimator 218 calculates three Rᵣₛ estimates: an MWLS estimate 218₀ using a 0^{th}-order polynomial (M=0, i.e. Pₘᵤₛ(t) is modeled as a constant); an MWLS estimate 218₁ using a 1^{st}-order polynomial (M=1, i.e. Pₘᵤₛ(t) is modeled as a linear function of t); and an MWLS estimate 218₂ using 2^{nd}-order polynomial (M=2, i.e. Pₘᵤₛ(t) is modeled as a quadratic function of t). The MWLS formulation for each MWLS estimator 218₀, 218₁, 218₂ is listed in the righthand box of FIG. 5 as well as in Table 1 below.

TABLE 1
Rᵣₛ estimator formulations for each Pₘᵤₛ(t) model

| | Pₘᵤₛ | Moving Window Least Squares |
|---|---|---|
| | Model | Estimator |
| 0^{th} | Pₘᵤₛ(t) = | \tilde{P}_{y}(t) ≅ Rᵣₛ\grave{V}(t) + Pₘᵤₛ(t) |
| Order | a₀ | [\tilde{R}_{rs,0}, a₀]^{T} = (X^{T} X)⁻¹X^{T}Y |
| | | Y = [\tilde{P}_{y}(t), \tilde{P}_{y}(t − 1), . . . , \tilde{P}_{y}(t − L + 1)]^{T} |
| | | X = [x(t), x(t − 1), . . . , x(t − L + 1)]^{T} |
| | | x(t) = [\grave{V}(t), 1]^{T} |
| | | δ_{\tilde{R}}^{_{rs,0}} = (X^{T} X)⁻¹(1, 1) * δ_{Py} |
| 1^{st} | Pₘᵤₛ(t) = | \tilde{P}_{y}(t) ≅ Rᵣₛ\grave{V}(t) + Pₘᵤₛ(t) |
| Order | a₀ + a₁t | [\tilde{R}ᵣₛ,₁,a, a₀, a₁]^{T} = (X^{T} X)⁻¹X^{T}Y |
| | | Y = [\tilde{P}_{y}(t), \tilde{P}_{y}(t − 1), . . . , \tilde{P}_{y}(t − L + 1)]^{T} |
| | | X = [x(t), x(t − 1), . . . , x(t − L + 1)]^{T} |
| | | x(t) = [\grave{V}(t), 1, t]^{T} |
| | | δ_{\tilde{R}}^{_{rs,1}} = (X^{T} X)⁻¹(1, 1) * δ_{Py} |
| 2^{nd} | Pₘᵤₛ(t) = | \tilde{P}_{y}(t) ≅ Rᵣₛ\grave{V}(t) + Pₘᵤₛ(t) |
| Order | a₀ + a₁t + | [\tilde{R}ᵣₛ,₂, a₀, a₁, a₂]^{T} = (X^{T} X)⁻¹X^{T}Y |
| | a₂t² | Y = [\tilde{P}_{y}(t), \tilde{P}_{y}(t − 1), . . . , \tilde{P}_{y}(t − L + 1)]^{T} |
| | | X = [x(t), x(t − 1), . . . , x(t − L + 1)]^{T} |
| | | x(t) = [\grave{V}(t), 1, t, t²]^{T} |
| | | δ_{\tilde{R}}^{_{rs,2}} = (X^{T} X)⁻¹(1, 1) * δ_{Py} |

With continuing reference to FIG. 5, the three Rᵣₛ(t) estimates output by the respective MWLS operations 218₀, 218₁, 218₂ are combined together by a combining operation 219 to produce the final MWLS estimate, \tilde{R}ᵣₛ(t). The combining operation 219 may use various combinational techniques, such as a maximum ratio combination operation or a minimum variance selection combination. The maximum ratio combination employed by the illustrative combiner 219 assigns the largest weight to the estimate with the least estimation variance (e.g. the one with the best polynomial order) so that the one with the best polynomial order will dominate the Rᵣₛ estimation output. The MWLS 218 also calculates the variance of \tilde{R}ᵣₛ(t), δ_{\tilde{R}}_{ᵣₛ}.

With returning reference to FIG. 2, in the case of the Rᵣₛ estimator 134 only the Rᵣₛ(t) estimate, \tilde{R}ᵣₛ (t), output by the MLS operation 218 is retained while the other estimation outputs (e.g. Pₘᵤₛ(t) polynomial coefficients) are discarded. In the final stage of the Rᵣₛ estimator 134, a Kalman filter 220 is applied to further improve the Rᵣₛ estimation output by the MWLS 218. The Kalman filter 220 is suitably similar to the Kalman filter 212 described above with respect to the Eᵣₛ estimator 132. The Kalman filter 220 for the Rᵣₛ estimator 134 can be designed to reduce the MWLS estimation noise based on the following assumptions: (1) a state process equation where Rᵣₛ changes slowly and can be modelled as a random walk, i.e. Rᵣₛ(t)=Rᵣₛ(t−1)+ω_{R}(t), ω_{R}(t−1)˜N(0,δ_{R}); and (2) an observation equation where the MWLS estimate \tilde{R}ᵣₛ(t) can be modelled as \tilde{R}ᵣₛ(t)=Rᵣₛ(t)+ω_{LR}(t), where ω_{LR} (t)˜N(0, δ_{\tilde{R}}_{ᵣₛ}(t)). A standard Kalman filter can be implemented with A=1, B=0, Q=δ_{R}, H=1, and R=δ_{\tilde{R}rs}(t). Again, the Kalman filter has certain advantages, including computationally efficient implementation in the context of a sliding time window, intuitive operation and output of weighted averages. The parameter δ_{R} is an algorithm parameter that controls the average window length.

The output 222 of the Rᵣₛ(t) Kalman filter 220 is the Rᵣₛ estimate notated here as \hat{R}ᵣₛ(t)=Rᵣₛ(t)+ωᵣ(t). This output assumes that \tilde{R}ᵣₛ(t) is an unbiased estimate of Rᵣₛ(t), but has a noise term ωᵣ (t)˜N(0, δ_{\tilde{R}}_{ᵣₛ}).

With reference back to FIG. 2, in the final pass, once the Eᵣₛ and Rᵣₛ estimates are obtained by the respective estimators 132, 134, the Pₘᵤₛ(t) estimator 136 is applied to estimate Pₘᵤₛ (t). Using the previously estimated Rᵣₛ(t) and Cᵣₛ(t), a Pₘᵤₛ(t) computation 224 computes the \tilde{P}ₘᵤₛ(t) estimate according to:

\tilde{P}ₘᵤₛ(t)=P_{y}(t)−Êᵣₛ(t)V(t)−\hat{R}ᵣₛ(t)\dot{V}(t)

evaluated over the samples of P_{y} (t), \dot{V}(t), and (via integrator 122) V(t) in the time window 130. Said another way, \tilde{P}ₘᵤₛ(t)=P_{y} (t)−\hat{R}ᵣₛ\dot{V}(t)−Êᵣₛ V(t) is evaluated in the time window 130 of the MWLS. To remove high-frequency noise in \tilde{P}ₘᵤₛ(t), an optional low-pass filter 226 can be used to further improve the Pₘᵤₛ(t) estimate. Additionally or alternatively, physiological knowledge of the Pₘᵤₛ(t) waveform can be infused to further improve the Pₘᵤₛ(t) estimation.

In the illustrative embodiments, the respiratory elastance (or compliance) estimator 132 is applied first, followed by the respiratory resistance estimator 134 and finally the respiratory muscle pressure estimator 136. However, it is contemplated to estimate the respiratory resistance first, followed by estimation of the respiratory elastance or compliance (that is, to reverse the order of the estimators 132, 134). In such a variant embodiment, the second (Eᵣₛ) estimator would suitably include an Rᵣₛ cancellation operation analogous to the operation 216 of the illustrative embodiment. Regardless of the order of estimation of Eᵣₛ (or Cᵣₛ) and Rᵣₛ, it will be appreciated that the final Pₘᵤₛ(t) estimator 136 could optionally be omitted if Pₘᵤₛ(t) and WoB (computed therefrom by integrator 140) are not used.

If the respiratory elastance (or compliance) and/or resistance are displayed on the display component 110 of the ventilator 100, these values may optionally also be displayed with their respective uncertainty metrics, for example expressed in terms of the δ or ω statistics described herein or functions thereof. While these or other respiratory parameters are described as being displayed on the display component 110 of the ventilator 100 in the illustrative examples, it will be appreciated that such values may additionally or alternatively be displayed on a bedside patient monitor, at a nurses' station computer, and/or may be stored in an Electronic Health Record (EHR) or other patient data storage system, or so forth. The illustrative respiratory system analyzer 120 is suitably implemented via the microprocessor of the ventilator 100; however, the respiratory system analyzer 120 could additionally or alternatively be implemented via a microprocessor of a bedside patient monitor or other electronic data processing device. The disclosed respiratory system analyzer functionality may also be embodied by a non-transitory storage medium storing instructions that are readable and executable by such a microprocessor or other electronic data processing device to perform the disclosed functionality. By way of example, the non-transitory storage medium may, for example, include a hard disk or other magnetic storage medium, optical disk or other optical storage medium, flash memory or other electronic storage medium, various combinations thereof, or so forth.

As previously noted, in addition to displaying one or more of the estimated values (e.g. one or more of the values Êᵣₛ(t), Ĉᵣₛ(t)=1/Êᵣₛ, \hat{R}ᵣₛ(t), \hat{P}ₘᵤₛ(t) optionally with its statistical uncertainty) as a real-time value, trend line or so forth, in another illustrative application the \hat{P}ₘᵤₛ(t) waveform may be used to synchronize the positive pressure applied by the ventilator 100 with respiratory effort expended by the patient 102, so as to reduce patient-ventilator dyssynchrony. In this application, the positive air pressure applied by the ventilator 100 is adjusted, e.g. increased or decreased, in synch with increasing or decreasing magnitude of \hat{P}ₘᵤₛ (t). In another control application, the WoB output by the integrator 140 may be used as a feedback signal for control of the ventilator 100. In general, the positive pressure applied by the ventilator 100 should increase with increasing measured WoB output by integrator 140, and this increased mechanical ventilation should result in a consequent reduction in patient WoB until the setpoint WoB is reached. As illustration, a proportional and/or derivative and/or integral controller (e.g. PID controller) may be used for this feedback control with the WoB signal from the integrator 140 serving as the feedback signal, a target WoB serving as the setpoint value, and the positive pressure being the controlled variable.

The respiratory system analyzer 120 has been tested with simulated data and with pig respiratory data, and the results show the analyser 120 can provide comparable results to invasive solutions and is stable under different ventilator settings, including low PSV settings. The analyzer 120 provides various benefits, including (but not limited to): providing real-time data (with a lag of a few seconds or less); sample-by-sample estimation (if successive windows overlap and are spaced by a single sample); tailorable trade-off between computational complexity and temporal resolution (faster computation by larger spacing between possibly overlapping windows traded off against reduced temporal resolution); rapid convergence (within 10 breaths in some tests) providing low initiation time; stability against unexpected disturbances; good computational efficiency employing, for example, efficient pseudo-inverse (L×4) matrix computation (where L is the window size, e.g. 60-90 samples in some suitable embodiments); and low memory requirements (storing the data for the current time window, around 60-90 samples for some embodiments).

As a further advantage, the respiratory system analyzer 120 suitably estimates the elastance or compliance Eᵣₛ(t), resistance Rᵣₛ(t), and respiratory muscle pressure Pₘᵤₛ(t) without receiving as input the respiratory phase or respiratory rate, and without making any a priori assumptions about these parameters (other than that Eᵣₛ and Rᵣₛ are assumed to be constant within any given time window of the MWLS estimation). The respiratory system analyzer 120 suitably operates only on the measured air pressure P_{y}(t) and air flow \dot{V}(t) along with V(t)=∫\dot{V}(t)dt which is derived by integrating \dot{V}(t) over time.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A medical ventilator device comprising:
a ventilator configured to deliver ventilation to a ventilated patient;
a pressure sensor configured to measure the airway pressure P_{y}(t) of the ventilated patient;
an air flow sensor configured to measure the air flow \dot{V}(t) into and out of the ventilated patient; and
a respiratory system analyzer comprising a microprocessor configured to estimate respiratory parameters of the ventilated patient using moving time window least squares (MWLS) estimation including (i) respiratory system elastance or compliance (Eᵣₛ or Cᵣₛ), (ii) respiratory system resistance (Rᵣₛ), and (iii) respiratory muscle pressure (Pₘᵤₛ(t)).

2. The medical ventilator device of claim 1 wherein the MWLS estimation in cludes, for each time window of the MWLS estimation, performing the following operations in order:
(1) estimating one of (i) elastance or compliance and (ii) resistance;
(2) estimating the other of (i) elastance or compliance and (ii) resistance using the estimated value from operation (1); and
(3) estimating respiratory muscle pressure using the values estimated in operations and.

3. The medical ventilator device of claim 2 wherein the operation estimates elastance or compliance and the operation estimates resistance using the estimated value of elastance or compliance from operation.

4. The medical ventilator device of claim 2 wherein the operation optimizes elastance Eᵣₛ, resistance Rᵣₛ, and the difference ΔPₘᵤₛ of the respiratory muscle pressure Pₘᵤₛ of the equation:
ΔP_{y}(t)=RᵣₛΔ\dot{V}(t)+EᵣₛΔV(t)+ΔPₘᵤₛ
with respect to the measured values of P_{y}(t) and \dot{V}(t) in the time window of the MWLS where V(t)=∫\dot{V}(t)dt and ΔP_{y}(t)=P_{y}(t)−P_{y}(t−1) and Δ\dot{V}(t)=\dot{V}(t)−\dot{V}(t−1) and ΔV(t)=V(t)−V(t−1).

5. The medical ventilator device of claim 4 wherein the operation optimizes the respiratory muscle pressure Pₘᵤₛ(t) and one of elastance Eᵣₛ and resistance Rᵣₛ of the equation:
P_{y}(t)=Rᵣₛ\dot{V}(t)+EᵣₛV(t)+Pₘᵤₛ(t)
with respect to the measured values of P_{y}(t) and \dot{V}(t) in the time window of the MWLS with the estimated value from operation held fixed and Pₘᵤₛ(t) modeled by a parameterized function of time.

6. The medical ventilator device of claim 5 wherein Pₘᵤₛ(t) is modeled by a polynomial function of time.

7. The medical ventilator device of claim 6 wherein operation is repeated with Pₘᵤₛ(t) modeled by zeroeth, first, and second order polynomial functions of time and the optimized elastance Eᵣₛ or resistance Rᵣₛ of the three repetitions are combined.

8. The medical ventilator device of claim 5 wherein the operation estimates respiratory muscle pressure as P_{y}(t)−\hat{R}ᵣₛ\dot{V}(t)−ÊᵣₛV(t) in the time window of the MWLS where \hat{R}ᵣₛ and Êᵣₛ are estimated values from operations and.

9. The medical ventilator device of claim 2 wherein one or both of the operations and includes applying a Kalman filter to the estimated value.

10. The medical ventilator device of claim 9 wherein one or both of the operations and further includes generating an uncertainty metric for the estimated value based on a noise variance of the Kalman filter.

11. The medical ventilator device of claim 1 further comprising:
a display configured to display one or more of the respiratory parameters of the ventilated patient estimated by the respiratory system analyzer.

12. The medical ventilator device of claim 1 wherein the ventilator is programmed to adjust positive air pressure output by the ventilator in synch with increasing or decreasing magnitude of the respiratory muscle pressure (Pₘᵤₛ(t)) in order to reduce patient-ventilator dyssynchrony.

13. The medical ventilator device of claim 1 wherein:
the respiratory system analyzer is configured to estimate a work of breathing (WoB) as WoB=∫Pₘᵤₛ(t)dV(t) where Pₘᵤₛ(t) is the respiratory muscle pressure as a function of time estimated using the MWLS estimation; and
the ventilator is programmed to control mechanical ventilation provided by the ventilator to maintain the estimated WoB at a setpoint WoB value.

14.-19. (canceled)

20. A non-transitory storage medium storing instructions readable and executable by an electronic data processing device to perform a method operating on measurements of airway pressure P_{y}(t) and air flow \dot{V}(t) of a patient on a ventilator, the method including:
applying moving window least squares (MWLS) estimation to estimate (i) respiratory system elastance Eᵣₛ, (ii) respiratory system resistance Rᵣₛ, and (iii) respiratory muscle pressure Pₘᵤₛ(t), wherein:
the MWLS estimation (i) comprises fitting:
ΔP_{y}(t)=RᵣₛΔ\dot{V}(t)+EᵣₛΔV(t)+ΔPₘᵤₛ
to ΔP_{y}(t) to obtain values for Eᵣₛ, Rᵣₛ, and ΔPₘᵤₛ, where ΔP_{y}(t) is a difference signal of the measured airway pressure, Δ\dot{V}(t) is a difference signal of the measured air flow, ΔV(t) is a difference signal of respiratory system air volume V(t)=∫\dot{V}(t)dt, and ΔPₘᵤₛ is a constant, and
the MWLS estimation (ii) comprises fitting:
P_{y}(t)=Rᵣₛ\dot{V}(t)+EᵣₛV(t)+Pₘᵤₛ(t)
to obtain values for Rᵣₛ and Pₘᵤₛ(t), where Eᵣₛ is set the value determined in the MWLS estimation (i) and with Pₘᵤₛ(t) is approximated as a parameterized function, and
the MWLS estimation (iii) comprises evaluating:
Pₘᵤₛ=P_{y}(t)−Rᵣₛ\dot{V}(t)−EᵣₛV(t)
where Eᵣₛ is set the value determined in the MWLS estimation (i) and Rᵣₛ is set the value determined in the MWLS estimation (ii).

21. The non-transitory storage medium of claim 20 wherein the respiratory system elastance Eᵣₛ is represented in the MWLS estimation operations as a respiratory system compliance Cᵣₛ=1/Eᵣₛ.
